(19) United States
(12) Patent Application Publication
(10) Pub. No.: US 2005/0228236 A1
(43) Pub. Date: Oct. 13, 2005

(54) Method and apparatus for assessing psychiatric or physical disorders

(75) Inventors: Joachim Diederich, Sohar, Oman (DE); Peter Yellowlees, Sacramento, CA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: THE University of Queensland, ST Lucia (AU)

(21) Appl. No.: 10/530,155

(22) PCT Filed: Oct. 3, 2003
(86) PCT No.: PCT/AU03/01307

§ 371 Date: Jun. 2, 2005

(30) Foreign Application Priority Data
Oct. 3, 2002 (AU) 2002-951811
Mar. 10, 2003 (AU) 2003901081

(51) Int. Cl. A61B 5/00

(52) U.S. Cl. 600/300; 128/920; 128/925

(57) ABSTRACT

A method of assessing the psychological or physiological state by analyzing language cues captured from a patient. The language cues may be semantic cues (speech or written text) or visual cues (expression or body language). Key features are extracted from the language cues and compiled into a data file which is submitted to one or more pre-taught machine learning algorithms. The output of the machine learning algorithms are combined to determine the psychological or physiological state of the patient. The method of teaching the machine learning algorithms is also described. In the preferred form there are three machine learning algorithms including a support vector machine, a decision tree learning algorithm and a neural network.

This invention relates to a method and apparatus for assessing psychiatric or physical disorders. In particular It relates to the classification of language cues as an indicator of the psychological or physiological state of a person.

### BACKGROUND TO THE INVENTION

At least 3% of the world population suffers from severe mental health problems including depression and schizophrenia. Mental health conditions such as schizophrenia, depression, etc are difficult to diagnose and treat. The success of treatment is enhanced if an early diagnosis is possible. Unfortunately, patients often do not seek treatment until the indicators of a mental health problem are pronounced. By the time treatment is sought the problem is chronic.

The known methods of assessing mental health conditions are subjective and rely upon both the skill of the clinician and the honesty of responses of the patient. This latter point is particularly difficult to achieve since patients often minimize or disguise their symptoms and hence make accurate diagnosis difficult.

It is known to use support vector machines (SVMs) for identification of the author of a document and for face detection and recognition. The use of SVM was first described in: B. E. Boser, I. M. Guyon, and V. N. Vapnik. A training algorithm for optimal margin classifiers. In D. Haussler, editor, 5th Annual ACM Workshop on COLT, pages 144-152, Pittsburgh, Pa., 1992. ACM Press.

SVMs have been used for text analysis: Joachims, T.: “Text Categorization with Support Vector Machines: Learning with Many Relevant Features”, in Proceedings of the Tenth European Conference on Machine Leaming (ECML '98), Lecture Notes in Computer Science, Number 1398 (pp. 137-142), 1998. SVMs have also been used for face detection: Osuna, E.; Freund, R.; Girosi, F.: Training Support Vector Machines: An application to face detection. Proc. IEEE Computer Vision and Pattern Recognition, 130-136, 1997. In: Yang., M.-H.; Kriegman, D. J.; Ahuja, N.: Detecting Faces in Images: A Surevy. IEEE Transactions on Pattern Analysis and Machine Intelligence. Vol. 24, No. 1, 34-58, 2002.

An ideal screening tool would be one that was an objective system that can operate without causing changes in, or influencing the behavior of the patient.

Unsuccessful attempts have been made to achieve this goal. One such attempt is described in International Patent Application number PCT/US96/12177 filed in the name of Horus Therapeutics Inc. This document describes a method of diagnosing a disease by collecting data about a patient into a data file and submitting the data file to a trained neural network. The neural network is trained by submitting data files from patients that have been diagnosed so that the neural network “learns” the correlations between the data files and various health conditions.

The Horus invention is limited to physiological disorders, such as osteoporosis and cancers. The invention focuses on the use of “biomarkers”, defined as quantifiable signs, symptoms and/or analytes in biological fluids and tissues. The biomarkers from patients (humans or animals) with known conditions are used to train the neural networks which are then used to diagnose biomarkers from patients with unknown conditions. There is no disclosure or suggestion of the use of language cues, either semantic or visual.

Horus Technologies Inc only teach the use of neural networks for diagnosing physiological disorders from biomarker data. It does not disclose the use of language cues nor does it disclose the diagnosis of psychological disorders.

Reference may also be had to a patent application by Dendrite Inc, filed as International Patent Application number PCT/US98/05531 titled Psychological and Physiological State Assessment System Based on Voice Recognition and it's Application to Lie Detection.

The patent application describes a method and apparatus for assessing the psychological and physiological state of a subject by comparing the speech of the subject with a stored knowledge base.

The spoken words are recorded, digitised and analysed to extract a time-ordered series of frequency representations. The frequency referred to is the audio frequency and not the frequency of occurrence of any particular word or phrase.

The invention is based upon the construction of a knowledge base that correlates speech parameters with psychological and/or physiological state. The knowledge base is constructed statically rather than using dynamic machine learning processes. The citation does not disclose the use of machine learning algorithms.

The citation describes an entirely aural process that extracts frequency parameters from the spoken word. There is no suggestion of using language cues.

International Patent Application number PCT/AU 01/00535, filed jointly by CSIRO, Unisearch and the University of Queensland, is titled Computer Diagnosis and Screening of Psychological and Physical Disorders. This document describes a method of diagnosing psychological and/or physical disorders by computer processing temporal data recorded for a subject over a predetermined time interval to extract indicators (such as degree of change over time) and correlating the indicators with a knowledge base of data to determine a disorder.

The specification provides a description of one embodiment of the invention where changes in facial expression over time are used as an indicator of melancholic depression. The specification does not disclose the use of machine learning algorithms nor the use of language as distinct from speech.

The prior art mentioned does not teach an objective system that can assess the psychiatric or physiological state of a patient

### DISCLOSURE OF THE INVENTION

In one form, although it need not be the only or indeed the broadest form, the invention resides in a method of assessing a psychological or physiological state including the steps of:
capture language cues that are indicative of the psychological or physiological state of a patient;
analyze the language cues to determine key features;
produce a data file containing data based upon the key features;
submit the data file to one or more pre-taught machine learning algorithms; and
combine output of the machine learning algorithms to determine the psychological or physiological state of the patient.

The language cues may suitably be semantic cues or visual cues. The semantic cues may be obtained directly from text prepared by the patient or from speech that is converted to text. Visual cues may include body language such as facial expression or other body movements.

In the case of semantic cues the step of analyzing language cues may include extracting key features by analyzing a text sample to determine a frequency of occurrence of words, syllables, phonemes or other symbols. For visual cues the step may include capturing a sequence of images or a video sample and analyzing the changes in areas of interest over time to extract key features.

The data file may be based on pre-processing steps and transformations of data.

The invention may further include the preliminary steps of teaching the machine learning algorithms by:

combining language cues with classes of psychological or physiological disorders and symptom severity derived from clinical trials and clinical assessments to form the data file;

submitting the data file to the machine learning algorithms; and translating the internal representation of the machine learning algorithms into symbolic rules.

Suitably the machine learning algorithms include a support vector machine, a decision tree learning algorithm, and a neural network.

Suitably the invention may also include a learning method in which language cues from patients known to have health problems and patients known not to have health problems are analyzed. In addition to the language cues, an expert-defined health related category must be provided for learning purposes. This category can be discrete (presence or absence of the expert-defined health problem) or it can be a ranking on a given scale representing the severity of the health problem. An expert ranking of language cues must be available for learning purposes if the invention is to operate in ranking mode.

In a further form the invention resides in a method of generating categories for psychological or physiological conditions including the steps of:
filtering a collection of expert descriptions of psychological or physiological conditions with a stoplist;
for each expert description, constructing a list of frequently occurring descriptive terms;
forming an intersection of the lists of frequently occurring descriptive terms;
submitting the expert descriptions to one or more machine learning algorithms;
using the intersection as the targets for machine learning; and
extracting internal representations of the machine learning algorithms as categories for psychological or physiological conditions after machine learning has been completed.

The method may further include the step of expanding the list with synonyms of the frequently occurring descriptive terms.

The expert descriptions may conveniently be obtained from expert psychiatrists or other, experienced health practitioners. A diagnostic report generated routinely by the psychiatrist is most suitable.

In a further form the invention resides in an apparatus for diagnosing or assessing a psychological or physiological state of a patient comprising:
means for capturing language cues;
a processor programmed to analyse the language cues and compile a data file;
one or more machine learning algorithms programmed in the processor and producing an output from the data file;
means for combining the outputs to produce an indicator of psychological or physiological state; and
display means adapted to display the psychological or physiological state of the patient.

### BRIEF DESCRIPTION OF THE DRAWINGS

To assist in understanding the invention, preferred embodiments will be described with reference to the following figures in which:

FIG. 1 shows a flowchart of a method of assessing health;

FIG. 2 shows a flowchart of a learning phase for speech/text that is preliminary to assessing health;

FIG. 3 shows a flowchart of a learning phase for image/video that is preliminary to assessing health;

FIG. 4 shows a block diagram of an apparatus for working the method;

FIG. 5a shows a sample of text from control subjects;

FIG. 5b shows sample of text from patients diagnosed with schizophrenia;

FIG. 6a shows sample of text from patients diagnosed as manic;

FIG. 6b shows a sample of text from control subjects;

FIG. 7 shows a sample of a word frequency table;

FIG. 8 shows a preprocessed text block formed from the sample texts;

FIG. 9 shows a decision tree learning file derived from the data of FIG. 8;

FIG. 10 shows decision tree learning results;

FIG. 11 shows a set of sample images; and

FIG. 12 shows the sample images of FIG. 11 after preprocessing.

### DETAILED DESCRIPTION OF THE DRAWINGS

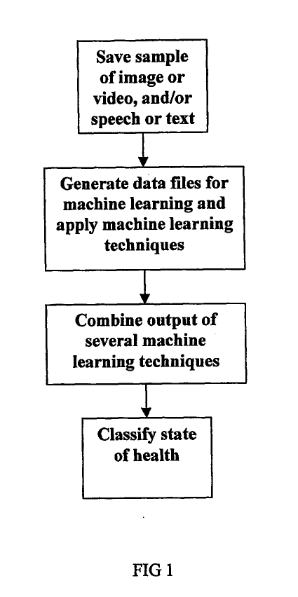

Referring to FIG. 1, there is shown a flowchart outlining the steps of a method for assessing health. The first step of the method is to obtain language cues from a patient, which may be samples of text or speech to obtain semantic cues or images or video samples, including facial expressions or body movement, to obtain visual cues. The language cues will be indicative of the psychological or physiological state of the patient. Analysis of the language cues leads to an indicator of the psychological or physiological state and hence an assessment of health.

If a speech sample is obtained it is preprocessed into a text block using known speech to text translation algorithms. Examples for suitable systems are ISIP (Institute for Signal and Information Processing, Mississippi State University), Sphinx (Carnegie Mellon University) and commercial packages such as Dragon's “Naturally Speaking”.

The language cues are processed to produce a datafile for machine analysis. The data file is submitted to two or more machine learning techniques and the combination of the outputs of the machine learning techniques is obtained. Three machine learning techniques are used in a preferred form. A support vector machine is used as one of the machine learning techniques and decision tree learning and a neural network are the other two.

The combination of the output of the machine learning methods represents the diagnosis. These outputs are compared against psychiatric classification parameters and symptom severity measurements to validate them as diagnostic tools.

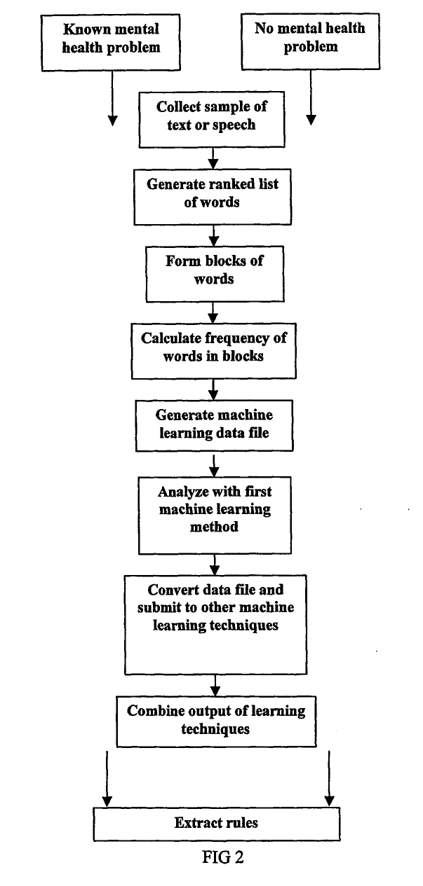

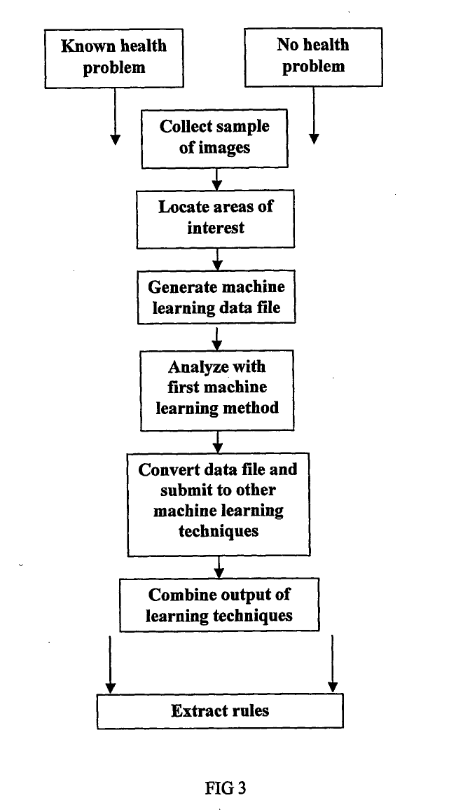

In order to work the invention in a diagnostic mode it must first be operated in a learning mode to build the association between the output and the language cues. The learning process for text and speech samples is shown in the flow chart of FIG. 2. The flowchart of FIG. 3 shows the analogous process for image and video samples.

The learning phase includes collecting language cue samples from patients known to have psychiatric or physiological disorders (these are marked as positive samples). Samples are also obtained from people who are known not to have the problem (these are marked as negative samples). A sufficiently large data set must be available to guarantee the statistical validity of the method.

If the intended use of the system is classification (diagnosis), mark language cue samples from patients with the expert-defined health problem as positive examples and all others as negative. If the intended use of the system is a ranking, obtain expert ranking with regard to the psychiatric or physiological disorder for language cue samples.

As shown in FIG. 2, a ranked list of words or symbols according to frequency is generated from the corpus of all samples obtained (positives and negatives). The words are then formed into blocks of words or symbols of user-determined length. For each block of words or symbols the frequency of occurrence of each word or symbol is recorded. The data may be normalised or otherwise transformed. This may include the exclusion of high-frequency words, stemming, the formation of Ngrams (combination of words), the use of TF/IDF (term frequency/inverse document frequency) calculations and other pre-processing techniques.

A data file is generated for submission to two or more machine learning algorithms. In the preferred form of the invention, one of these machine learning algorithms is a support vector machine (SVM) as described in B. E. Boser, I. M. Guyon, and V. N. Vapnik. A training algorithm for optimal margin classifiers. In D. Haussler, editor, 5th Annual ACM Workshop on COLT, pages 144-152, Pittsburgh, Pa., 1992. ACM Press.

The machine learning techniques can be applied in any order. In case of SVM learning, each row in the datafile represents an image or video sample in the case of visual language cues or a block of words in the case of semantic language cues. It includes the class label [1 if this sample is from a person with a health problem, −1 otherwise]. If the system is to produce a ranking, expert-ranking replaces the class label. This is followed by attribute-value pairs. Attributes are words represented by numbers (the ranking of the word in the corpus) plus the frequency of occurrence of the word in this block of text or elements of the images or video.

In the visual cue implementation, the elements are part of a face (identified by machine learning) that express a psychiatric or physical disorder, including extreme states of emotion: both sides of the mouth as well as the outside area of the eyes in addition to the area around both the eyes. The data may be normalized or otherwise transformed.

The data file is submitted to the SVM so that it “learns” the difference between positives and negatives. Once trained the SVM will generate an output for an unknown language cue that will be indicative of the presence or otherwise of the health problem.

During learning, the SVM adjusts parameters to approach the target outcome. The set of parameters that achieve the target outcome are saved in a model file. The model file is used to generate rules that become part of the diagnostic device.

The data file is translated to a suitable form for the second and subsequent machine learning algorithms. By way of example, the other two algorithms may be a decision tree algorithm (DT) and a neural network algorithm (NN): Tickle, A. B.; Andrews, R.; Golea, M.; Diederich, J.: The truth will come to light: directions and challenges in extracting the knowledge embedded within trained artificial neural networks. IEEE Transactions on Neural Networks 9 (1998) 6, 1057-1068. When translating the data file for use by the decision tree algorithm or the neural network, it may be necessary to limit the number of attributes.

As with the SVM, the outputs from the DT and the NN will be indicative of the presence or otherwise of a health problem in the language cue sample. The set of parameters (for example, weights in the case of the neural network) are used to generate rules that become part of the diagnostic device, as with the SVM rules discussed above. The rules (weights, parameters, etc) direct information flow through the machine learning algorithms in the diagnostic device.

The outputs can be combined in a variety of ways to achieve the best outcome. At the simplest level the outcomes may be combined in a simple vote. For instance, if two algorithms diagnose a problem and one does not, the outcome would be considered as positive with respect to that problem. Other combination techniques, such as weighted averages, would also be suitable. In such a case the weighting may be derived from the relative effectiveness of each algorithm of assessing a given health problem.

Once the invention has been trained to recognize the difference between positives and negatives, rules are extracted to be used as a possible input to the invention in the diagnostic (classification or ranking) mode. The rule extraction may be performed for the SVM, DT and NN. Rule extraction from the DT is built-in, rule-extraction from the SVM proceeds by applying decision tree learning to the inputs and outputs of the SVM, and rule-extraction from NN is using one of the methods in Tickle, A. B.; Andrews, R.; Golea, M.; Diederich, J.: The truth will come to light: directions and challenges in extracting the knowledge embedded within trained artificial neural networks. IEEE Transactions on Neural Networks 9 (1998) 6, 1057-1068.

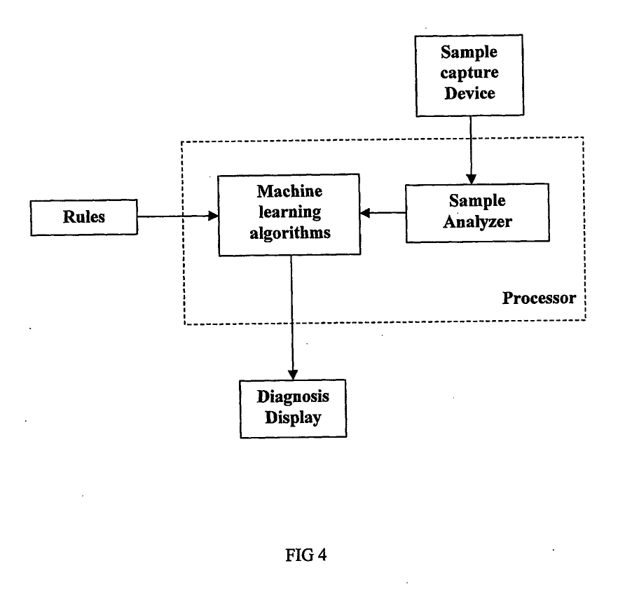

An apparatus suitable for working the method is depicted in FIG. 4. A sample capture device captures language cue samples from any suitable source. A text sample may be captured from an email, newsgroup message, letter, essay, poem, newspaper article, etc. If a voice sample is captured it is converted to a text sample using known voice to text translation algorithms. This may occur in the sample capture device or externally. Suitable voice samples maybe a telephone conversation, a public presentation, a clinical interview, etc. A sequence of images or video sample including facial expressions or body movement may be captured from TV, the Internet, multimedia data repositories etc.

The sample is passed to a processor that includes an analyzer that forms the data file. The data file may be generated in a number of different forms to suit the machine learning algorithms employed. The data file is then processed according to a rule set or using two or more machine learning algorithms. The rules may suitably be stored external from the processor.

The outputs from the algorithms are then combined. A diagnostic display, which may be graphic or text, is produced. The display may be visual or hard copy.

It will be appreciated that after successful completion of the learning phase the invention can be used to classify any language cue sample of minimal length into one or more health related categories, including depression, mania, etc. The method can be used to assess a health problem without the knowledge of the subject. This provides a completely objective assessment that cannot be biased by a patient.

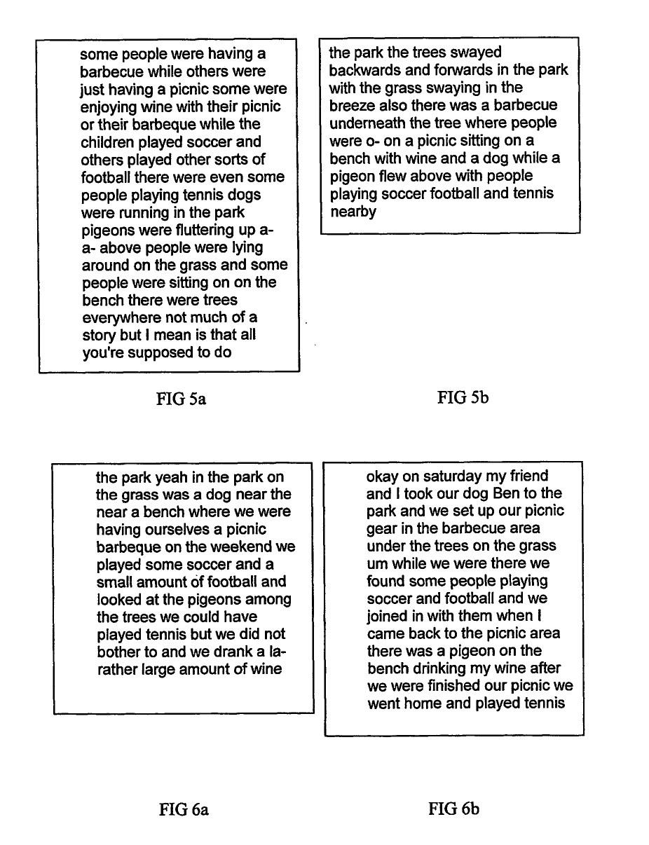

The effectiveness of the invention can be demonstrated in the following example of detection of schizophrenia. A small sample of 56 patients were tested. The patients comprised three groups: 31 with clinically diagnosed schizophrenia; 16 patients with clinically diagnosed mania; and 9 control subjects. Speech samples were collected from each patient using a structured narrative task. A typical block of narrative text from a patient in the schizophrenia group is shown in FIG. 5a with a corresponding control in FIG. 5b. Another block of control text is shown in FIG. 6a with text from a patient in the mania group in FIG. 6b.

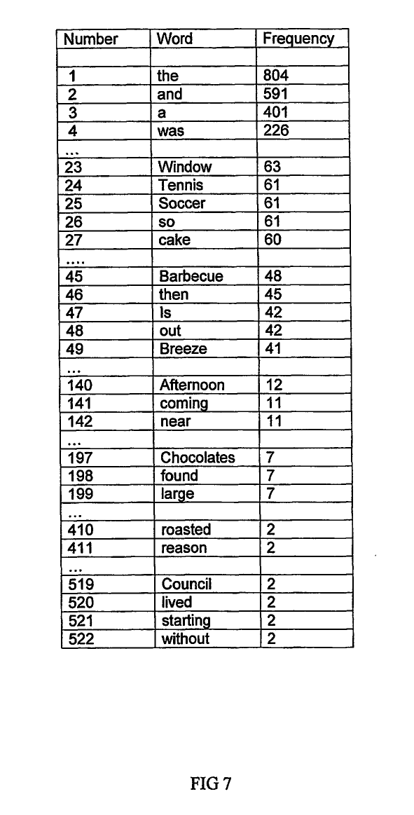

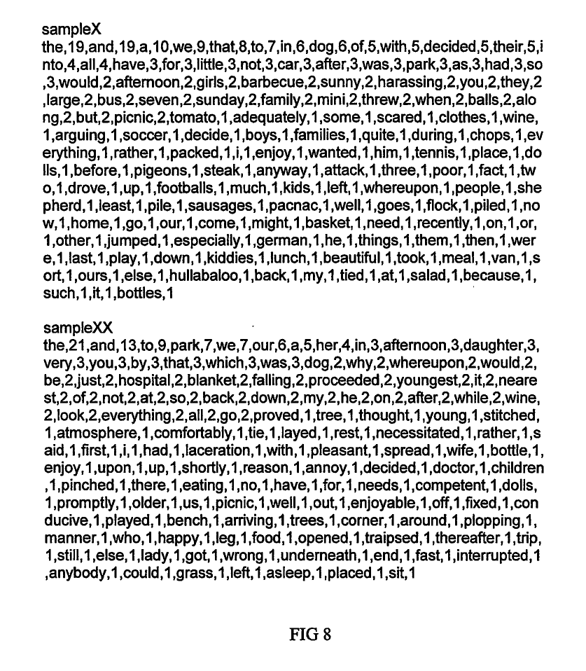

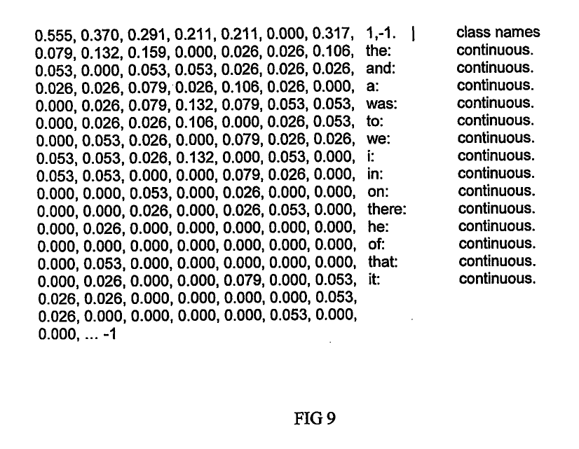

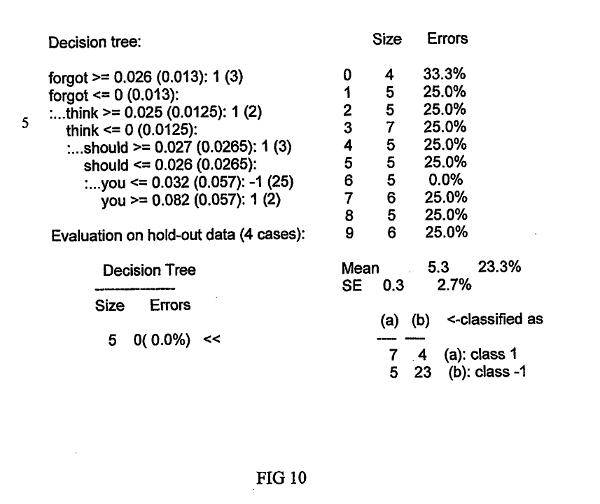

The frequency of occurrence of words in all the text samples is calculated and tabulated. A sample of the frequency table is shown in FIG. 7. Based upon the word frequency listing, each text sample is preprocessed into a block of words and frequencies, a shown in FIG. 8. These blocks are then transformed to data files for the machine learning techniques. A decision tree data file is shown in FIG. 9. The decision tree algorithm learning results are presented in FIG. 10. For this example a stoplist has been used to make presentation of results more tractable. A stoplist typically includes function words such as articles, pronouns and prepositions as well as other high-frequency words which are eliminated prior to processing to increase the explanatory power of the learning results.

Despite the use of a structured narrative task, the correlation of the test subjects to expert clinical diagnosis was about 82%. The use of unstructured text and larger samples will further improve the correlation.

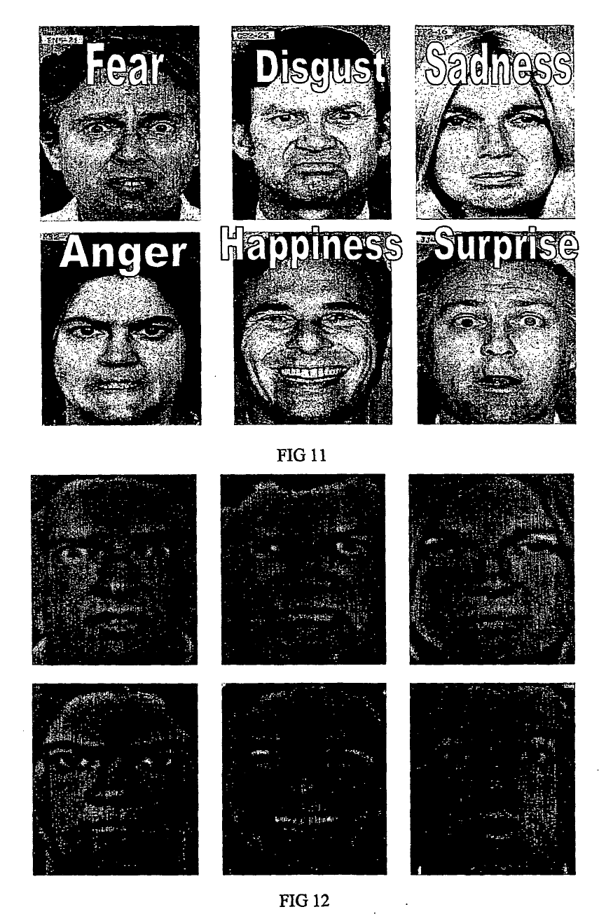

To exemplify the use of the invention with image samples the processing steps for the images shown in FIG. 11 are discussed below. FIG. 11 shows six typical facial expressions which could be used in the invention. As with the text/speech embodiment, preprocessing of the images is required. The preprocessed images are shown in FIG. 12.

Each image is pixilated and the intensity in each pixel is recorded. Images are converted to grey-scale and local response functions (kernel functions) are used to (1) determine regions of interest and (2) map regions of interest to output categories or rankings.

In another example, 72 diagnostic reports were assessed. The reports were modified by removing header and footer information (names, addresses, compliments) and then a ranked list of n words was produced for each document, excluding words in a stop list of the 6500 most spoken words in the English language. The intersection of the ranked words was formed as described above. Several cluster algorithms were applied to the ranked word lists and the outputs of the cluster algorithms were combined and merged. The resultant final clusters provided new diagnostic categories.

It will further be appreciated that the invention is not limited to the diagnosis of a health problem when one is suspected. The invention can be used in a screening application to monitor the health of groups of subjects, for example key decision makers in government jobs. In particular, the method can be embedded in a search engine that ranks documents, audio files, images and video files with regard to psychiatric or physical disorders for a given combination of search items.

In the search engine application the method can be used to extract information from a corpus of documents, such as the Internet, based on psychological state. A conventional search engine can find documents or images that satisfy a given criteria such as (president and (microsoft or windows)). The invention can add a psychological dimension to the search engine. For a given combination of key words, the ranking of returned documents is determined by the psychological state expressed in the texts. An expert ranking of documents is required for learning purposes. The information is then assessed in the manner described above to determine the psychological state of the author.

There are various language cues for different mental health problems, for example:

Depression—slowed movement of facial and truncal muscles groups, greater time latency between words and movements, impoverished or reduced vocabulary, depressive typology;

Schizophrenia—abnormal movements, turning of head in response to hallucinations, occasional ticks and jerks, spasms, abnormal involuntary grimaces and tongue movements, scared look, wide eyes, abnormal speech content, disorganized speech patterns, paranoid language, lack of coherent or logical sentences;

Dementia—flatness and vacancy, lack of emotional movement, stretched and flat skin, reduced or impoverished vocabulary, impoverished speech pattern, childlike vocabulary, repetitive, lack of consistency and continuity.

It will be appreciated that there are common indicators between these three conditions. The invention is able to distinguish between these conditions and provide improved diagnosis compared to known techniques, which can confuse diagnosis of these conditions.

Another benefit of the invention is the ability to define new diagnostic categories. Traditional diagnostic categories are “fuzzy” and ill-defined. Many practitioners view the categories as simplifications of complex psychological or physiological states.

As part of one form of the invention, text mining, and in particular text summarization, is used to generate suitable targets for machine teaming.

Prior to machine learning, several expert psychiatrists or other health practitioners are asked to nominate a condition/disorder with symptoms that may be expressed in speech/text/facial expression or human movement. This condition may not be part of an existing assessment scale or may be a combination of known classes of disorders.

The experts are asked to describe the condition on half a page or more. This textual description is then analyzed in one or more ways.

In one embodiment the following steps are taken:

(1) The textual descriptions are filtered by a stoplist (the Oxford list of the 6000 most frequent words in English or a shorter version). The stoplist may be edited: emotion words are excluded from the stoplist. Stemming may be used to make sure all forms of common words are eliminated.

(2) For each of the filtered documents, a list of the n most frequent words is formed.

(3) The intersection of all lists is formed (if there are fewer than k diagnostic descriptions, use words that occur in m or more of these texts). These are the targets for machine learning.

In an alternate embodiment, the following steps are taken

(1) The textual descriptions are filtered by a stoplist and Ngrams of content words are generated.

(2) A dictionary/lexicon (such as Wordnet) is used to search for synonyms. The list of Ngrams is expanded by inserting synonyms and forming new Ngrams. For each of the filtered documents, a list of the n most frequent Ngrams is formed.

(3) The intersection of all lists is generated (if there are fewer than k diagnostic descriptions, words that occur in m or more of these texts are used). These are the targets for machine learning.

Alternatively, full text summarisation is used and content words are filtered to generate targets.

The invention generates and diagnoses to fine-grained categories of psychiatric and physical diagnosis rather than the existing coarse-grained categories.

Throughout the specification the aim has been to describe the preferred embodiments of the invention without limiting the invention to any one embodiment or specific collection of features.

1. A method of assessing a psychological or physiological state including the steps of:
capture language cues that are indicative of the psychological or physiological state of a patient;
analyze the language cues to determine key features;
produce a data file containing data based upon the key features; submit the data file to one or more pre-taught machine learning algorithms; and
combine output of the machine learning algorithms to determine the psychological or physiological state of the patient.

2. The method of claim 1 wherein the language cues are semantic cues.

3. The method of claim 1 wherein the language cues are visual cues.

4. The method of claim 2 wherein the semantic cues are obtained directly from text prepared by the patient.

5. The method of claim 2 wherein the semantic cues are obtained from speech that is converted to text.

6. The method of claim 3 wherein the visual cues include body language such as facial expression or other body movements.

7. The method of claim 1 wherein the step of analyzing the language cues includes the step of extracting key features from semantic cues by analyzing a text sample to determine a frequency of occurrence of words, syllables, phonemes or other symbols.

8. The method of claim 1 wherein the step of analyzing language cues includes the step of extracting key features from visual cues by capturing a sequence of images or a video sample and analyzing the changes in areas of interest over time.

9. The method of claim 1 wherein the step of producing the data file further includes pre-processing steps and transformations of data.

10. The method of claim 9 wherein the pre-processing steps are selected from one or more of: exclusion of high frequency words; time frequency/inverse document frequency calculations; normalization; and translation to a form required for the one or more machine learning algorithms.

11. The method of claim 1 wherein the machine learning algorithms are selected from one or more of: a support vector machine; a decision tree learning algorithm; and a neural network.

12. The method of claim 1 further including the preliminary steps of teaching the machine learning algorithms by:
combining language cues with classes of psychological or physiological disorders and symptom severity derived from clinical trials and clinical assessments to form the data file;
submitting the data file to the machine learning algorithms; and
translating the internal representation of the machine learning algorithms into symbolic rules.

13. The method of claim 12 wherein the pre-taught machine learning algorithms are pre-taught by a learning method including analyzing language cues from patients known to have health problems and patients known not to have health problems.

14. The method of claim 12 further including the step of providing an expert-defined health related category for learning purposes.

15. The method of claim 12 further including the step of providing an expert-defined health related category for learning purposes wherein the expert-defined health related category is discrete.

16. The method of claim 12 further including the step of providing an expert-defined health related category for learning purposes wherein the expert-defined health related category is a ranking on a given scale representing the severity of the health problem.

17. The method of claim 12 further including the step of extracting internal representations of the machine learning algorithms as categories for psychiatric or physical conditions after machine learning has been completed.

18. A method of generating categories for psychological or physiological conditions including the steps of:
filtering a collection of expert descriptions of psychological or physiological conditions with a stoplist;
for each expert description, constructing a list of frequently occurring descriptive terms;
forming an intersection of the lists of frequently occurring descriptive terms;
submitting the expert descriptions to one or more machine learning algorithms;
using the intersection as the targets for machine learning; and
extracting internal representations of the machine learning algorithms as categories for psychological or physiological conditions after machine learning has been completed.

19. The method of claim 18 further including the step of expanding the list with synonyms of the frequently occurring descriptive terms.

20. The method of claim 18 wherein the expert descriptions are obtained from expert psychiatrists or other experienced health practitioners.

21. An apparatus for diagnosing or assessing a psychological or physiological state of a patient comprising:
means for capturing language cues;
a processor programmed to analyze the language cues and compile a data file;
one or more machine learning algorithms programmed in the processor and producing an output from the data file;
means for combining the outputs to produce an indicator of psychological or physiological state; and
display means adapted to display the psychological or physiological state of the patient.

22. A method of extracting information from a corpus of documents including the steps of:
analyzing the corpus of documents to extract information meeting determined content criteria;
capturing language cues from the extracted information that are indicative of the psychological state of an author of the extracted information;
analyzing the language cues to determine key features;
producing a data file containing data based upon the key features;
submitting the data file to one or more pre-taught machine learning algorithms;
combining output of the machine learning algorithms to determine the psychological state of the author; and
returning extracted information that meets a determined psychological state.
